(19) United States
(12) Patent Application Publication
(10) Pub. No.: US 2022/0175884 A1
(43) Pub. Date: Jun. 9, 2022

(54) MODULATION OF WNT SIGNALING IN AUDITORY DISORDERS

(71) Applicant: Surrozen Operating, Inc., South San Francisco, CA (US)

(72) Inventors: Sungjin LEE, Palo Alto, CA (US); Yang LI, Mountain View, CA (US); Wen-Chen YEH, Belmont, CA (US)

(21) Appl. No.: 17/438,806

(22) PCT Filed: Apr. 1, 2020
(86) PCT No.: PCT/US20/26227

§ 371 Date: Sep. 13, 2021

Related U.S. Application Data
(60) Provisional application No. 62/828,100, filed on Apr. 2, 2019.

(51) Int. Cl. A61K 38/18; A61K 38/30

(52) U.S. Cl. CPC A61K 38/1808; A61K 38/30; A61K 38/1825

(57) ABSTRACT

Methods of treating auditory disorders with modulators of the WNT signaling pathway are disclosed. Also provided are methods of administration and pharmaceutical compositions.

### CROSS REFERENCE TO RELATED APPLICATIONS

This application claims priority to U.S. Provisional Application No. 62/828,100, filed Apr. 2, 2019, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SRZN_015_01WO_ST25.txt. The text file is about 42 KB, created on Mar. 30, 2020, and is being submitted electronically via EFS-Web.

### FIELD OF THE INVENTION

The present invention provides WNT pathway modulators as a treatment for auditory disorders, in particular, for the regeneration of sensory hair cells of the inner ear.

### BACKGROUND OF THE INVENTION

Permanent damage to the hair cells of the inner ear results in sensorineural hearing loss, leading to communication difficulties in a large percentage of the population. Hair cells are the receptor cells that transduce the acoustic stimulus. Regeneration of damaged hair cells provides an avenue for the treatment of a condition that currently has no therapies other than prosthetic devices. Although hair cells do not regenerate in the mammalian cochlea, new hair cells in lower vertebrates are generated from epithelial cells, called supporting cells, that surround hair cells.

The prevalence of hearing loss after damage to the mammalian cochlea has been thought to be due to a lack of spontaneous regeneration of hair cells and/or neurons, the primary components to detect sound (Wong and Ryan (2015) Front Aging Neurosci. 7:58). Humans are born with about 15,000 inner ear hair cells, and hair cells do not regenerate after birth. Supporting cells, which surround hair cells in the normal cochlear epithelium, have the potential to differentiate into new hair cells in the neonatal mouse following ototoxic damage (Bramhall et al. (2014) Ear Hear 38(1)). Using lineage tracing, the new hair cells have been shown to arise from Lgr5-expressing inner pillar and third Deiters cells, and new hair cell generation has been shown to be incrementally increased by pharmacological inhibition of Notch (Bramhall et al. (2014) (supra), Mizutari et al. (2014) Frontiers in Pharmacology, 5:198). It has been postulated that the neonatal mammalian cochlea has some capacity for hair cell regeneration following damage alone (Cox et al. (2014) Development 141: 816-829) and that Lgr5-positive (Lgr5+) cells act as hair cell progenitors in the cochlea (Chai et al. (2011) J Assoc Res Otolaryngol. 12(4): 455-469, Shi et al. (2012) J Neurosci. 32(28): 9639-9648).

Currently, very few cases of hearing loss can actually be cured. Audiological devices such as hearing aids have limitations including the inability to improve speech intelligibility. Of those impacted by hearing impairments, less than 20 percent presently use hearing instruments. In cases of age-related, noise- or drug-induced auditory dysfunctions, often the only effective way to currently “treat” the disorder or reduce its severity is prevention: avoiding excessive noise and using ear protectors, practicing a healthy lifestyle, and avoiding exposure to ototoxic drugs and substances if possible.

Once the hearing loss has developed, people may use a hearing aid to correct the hearing loss. However, despite advances in the performance of these prostheses, they still have significant limitations. For example, hearing aids mainly amplify sound and cannot correct for suprathreshold or retrocochlear impairments such as impaired speech intelligibility, speech in noise deficits, tinnitus, hyperacusis, loudness recruitment and various other types of central auditory processing disorders. Hearing aids essentially amplify sounds, which stimulate unimpaired cells, but there is no therapy for aiding recovery of impaired cells or maximizing the function of existing unimpaired cells.

The present disclosure addresses the need for therapies for patients suffering from sensorineural hearing loss by providing therapeutic agents and methods for the regeneration of hair cells.

### BRIEF DESCRIPTION OF THE DRAWINGS

FIG. 1 is a graph showing relative gene expression of the indicated FZD, LRP5/6 and Axin2 genes in adult mouse cochlea. For each gene, the left two bars show expression in normal cochlea, and the two right bars show expression in damaged cochlea.

FIG. 2 is a graph showing stimulation of signaling in 293 STF reporter cells over-expressing FZD9 by the engineered FZD9-specific WNT agonist.

### SUMMARY OF THE INVENTION

The present invention is based, in part, upon the use of WNT agonists to regulate proliferation and differentiation of supporting cells into hair cells in the cochlea of the human ear.

In certain embodiments, the present invention provides a method of treating a subject suffering from an auditory disorder comprising administering to the subject an engineered WNT signaling modulator. In certain embodiments, the WNT signaling modulator is a WNT agonist, e.g., an engineered WNT agonist. In some embodiments, the WNT agonist is an engineered molecule comprising: a first domain that binds to one or more FZD receptors; and a second domain that binds to LRP5/6. In further embodiments, the WNT agonist is selected from the group consisting of an antibody or fragment thereof containing at least one epitope binding domain, a small molecule, an siRNA, and an antisense nucleic acid molecule. In some embodiments, the engineered WNT agonist comprises binding compositions that bind to one or more FZD receptors and binding compositions that bind to one or more LRP receptors. In other embodiments, the engineered WNT agonist comprises a tissue targeting molecule. In further embodiments, the tissue targeting molecule is an antibody or fragment thereof that binds to a tissue specific cell surface antigen. In some embodiments, WNT agonist is administered locally by injection into inner ear. In further embodiments, the WNT agonist is administered with another molecule selected from the group consisting of growth factors, HDAC inhibitors, and gamma-secretase inhibitors. In certain embodiments the auditory disease is hearing loss due to cochlear tissue damage. In further embodiments, the cochlear tissue damage is sensory hair cell loss.

In certain embodiments, the present invention provides a method of treating a subject suffering from an auditory disorder comprising administering to the subject a tissue-specific WNT signal enhancing molecule, e.g., an engineered tissue-specific WNT signal enhancing molecule. In some embodiments, the WNT signal enhancing molecule is an engineered molecule comprising: a first domain that binds to one or more E3 ubiquitin ligases; and a second domain that binds to a tissue specific receptor. In certain further embodiments, the E3 ubiquitin ligases are selected from the group consisting of Zinc and Ring Finger Protein 3 (ZNRF3) and Ring Finger Protein 43 (RNF43). In some embodiments, the first domain comprises an R-spondin (RSPO) polypeptide. In further embodiments, the RSPO polypeptide is selected from the group consisting of RSPO-1, RSPO-2, RSPO-3, and RSPO-4. In yet further embodiments, the RSPO polypeptide comprises a first furin domain and a second furin domain. In certain embodiments, the second furin domain is wild-type or is mutated to have lower binding to Leucine-rich repeat-containing G protein coupled receptors 4-6 (LGR4-6). In some embodiments, the WNT signal enhancing molecule incorporates a tissue targeting molecule. In further embodiments, the tissue targeting molecule is an antibody or fragment thereof that binds to a tissue specific cell surface antigen. In certain embodiments, the WNT agonist is administered locally by injection into inner ear. In further embodiments, the WNT agonist is administered with another molecule selected from the group consisting of growth factors, HDAC inhibitors, and γ-secretase inhibitors. In certain embodiments, the auditory disorder is selected from the group consisting of: hearing loss caused by exposure to loud noise, aging, ototoxicity, head trauma, virus infection, autoimmune inner ear disease, heredity, Meniere's disease, otosclerosis, tumors, and vestibular disorders, including vestibular hypofunction. In some embodiments, the auditory disorder is hearing loss due to cochlear tissue damage. In a further embodiment, the cochlear tissue damage is loss of sensory hair cells.

In certain embodiments, the present invention provides a method of treating a subject suffering from an auditory disorder comprising administering to the subject a WNT agonist, e.g., an engineered WNT agonist, and a tissue specific WNT signal enhancing combination molecule, e.g., an engineered tissue-specific WNT signal enhancing combination molecule (a “combination molecule”). In some embodiments, the tissue-specific WNT signal enhancing combination molecule comprises: the WNT agonist having at least one binding domain that binds to at least one FZD receptor and at least one binding domain that binds to at least one LRP receptor; and the tissue-specific WNT signal enhancing molecule comprising a first domain that binds to one or more E3 ubiquitin ligases; and a second domain that binds to a tissue specific receptor. In a further embodiment, the E3 ubiquitin ligases are selected from the group consisting of Zinc and Ring Finger Protein 3 (ZNRF3) and Ring Finger Protein 43 (RNF43). In still a further embodiment, the first domain comprises an R-spondin (RSPO) polypeptide. In yet a further embodiment, the RSPO polypeptide is selected from the group consisting of RSPO-1, RSPO-2, RSPO-3, and RSPO-4. In certain embodiments, the RSPO polypeptide comprises a first furin domain and a second furin domain. In a further embodiment, the second furin domain is wild-type or is mutated to have lower binding to Leucine-rich repeat-containing G protein coupled receptors 4-6 (LGR4-6). In some embodiments, the combination molecule incorporates a tissue targeting molecule. In yet further embodiments, the tissue targeting molecule is an antibody or fragment thereof that binds to a tissue specific cell surface antigen. In certain embodiments, the WNT agonist is administered locally by injection into inner ear. In further embodiments, WNT agonist is administered with another molecule selected from the group consisting of growth factors, HDAC inhibitors, histone acetyl transferase (HAT) agonists, DNA methyl transferase (DNMT) inhibitors, Atoh1 agonists, and Notch inhibitors such as γ-secretase inhibitors. In certain embodiments, the auditory disorder is hearing loss caused by exposure to any of: loud noise, aging, ototoxicity, head trauma, virus infection, autoimmune inner ear disease, heredity, Meniere's disease, otosclerosis, tumors, or vestibular disorders, including vestibular hypofunction. In some embodiments, the auditory disorder is hearing loss due cochlear tissue damage. In further embodiments, the cochlear tissue damage is loss of sensory hair cells.

### DETAILED DESCRIPTION

The mammalian hearing organ, the organ of Corti, is comprised of both mechanosensory hair cells (HCs) and nonsensory supporting cells (SCs), but the loss of HCs results in permanent hearing loss (Bohne et al. (1976) Trans Sect Otolaryngol Am Acad Ophthalmol Otolaryngol. 82:1); Hawkins et al. (1976) Acta Otolaryngol. 81(3-4):337-43; Kelley (2007) Proc Natl Acad Sci USA. 104(42):16400-1; Oesterle et al. (2008) J Assoc Res Otolaryngol. 9(1): 65-89. Embryonic, neonatal, and juvenile SCs can be converted to HCs by the forced expression of Atoh1 in vivo (Atkinson et al. (2014) PLoS ONE 9(7): e102077; Gubbels et al. (2008) Nature 455(7212):537-541; Izumikawa et al. (2008) Hear Res 240:52-56; Izumikawa et al. (2005) Nat Med 11:271-276; Kawamoto et al. (2003) J Neurosci 23:4395-4400; Kelly et al. (2012) Jaro-Journal of the Association for Research in Otolaryngology, 13 (4), 473-484; Kraft et al. (2013) Laryngoscope 123(4):992-9; Kuo et al. (2015) The Journal of Neuroscience 35(30):10786-10798; Liu et al. (2014) PLoS One 9(2):e89377; Liu et al. (2012) J Neurosci. 32(19): 6600-6610; Pan et al. (2013) Peripheral Nerve Injury and Neural Regeneration 8(17):1551-1559; Wu et al. (2013) J Biomed Nanotechnol. 9(10):1736-45. A clinical trial using Atoh1 gene therapy was approved by the FDA for HC regeneration (CGF166 by Novartis and GenVec). It was paused for further review of the trial's Data Safety Monitoring Board (GenVec Press Release Jan. 20, 2016) and continued afterwards (GenVec Press Release May 2, 2016). Referring to the regeneration in other systems, it is believed additional factors are required to facilitate more efficient and fully functional HCs. One example is that co-activation of canonical WNT signaling with Atoh1 overexpression increased the number of converted HCs in vivo by nearly 10-fold (Kuo et al. (2015) The Journal of Neuroscience 35(30):10786-10798). Other reports also say that inhibition of GSK3β, endogenous canonical WNT signaling inhibitory molecule, increased supporting cell proliferation, indicating that the effect is mediated by standard WNT signaling. After hair cell damaged by neomycin, an increase in WNT activity was also observed, which is consistent with an increase in proliferation observed in support cells (Head et al. (2013) Dev Dyn. 242:832-46). Furthermore, overexpression of Dkk1b, which is another endogenous inhibitory molecule of WNT signaling pathways, slowed the proliferative response to hair cell damage and suggests that at least WNT signaling is required for the mitosis stage of regeneration. Similarly, the WNT activators promote supporting cell proliferation and increase the number of hair cells formed (Head et al. (2013) supra; Jacques et al. (2014) Development 140:247).

Insights into the mechanisms of WNT action have emerged from several systems: genetics in Drosophila and Caenorhabditis elegans; biochemistry in cell culture and ectopic gene expression in Xenopus embryos. Many WNT genes in the mouse have been mutated, leading to very specific developmental defects. As currently understood, WNT proteins bind to receptors of the Frizzled family on the cell surface. Through several cytoplasmic relay components, the signal is transduced to beta-catenin, which then enters the nucleus and forms a complex with TCF to activate transcription of WNT target genes. Expression of WNT proteins varies, but is often associated with developmental process, for example in embryonic and fetal tissues.

The R-spondin family of ligands and leucine rich repeat (Lgr) G-coupled family of receptors, was recently linked to WNT signaling (Jin and Yoon (2012) Int. J. Biochem. Cell Biol. 44: 2278-2287). R-spondins can bind to three members of Lgr receptors to regulate the strength of Wnt signaling (de Lau et al., (2011) Nature 476:293-297). Specifically, R-spondins are ligands for Lgr4, 5 and 6, which represent a phylogenetic subgroup of Lgr receptors (de Lau et al., (2012) J. Pathol. 228:300-309). In most tissues where R-spondin and Lgr's have been studied with regards to WNT signaling, it has been found that they act to potentiate downstream WNT signaling (Schuijers and Clevers (2012) EMBO J. 31, 2685-2696).

Lgr5 is expressed across a diverse range of tissues and has been identified as a biomarker of adult stem cells in many tissues including, but not limited to, the gut epithelia (Barker et al. (2007) Nature 449:1003-1007), kidney, hair follicle, and stomach (Barker et al, (2010) Cell Stem Cell 6:25-36; Haegebarth and Clevers, (2009)). It was first published in 2011, that mammalian inner ear hair cells are derived from LGR5⁺ cells (Chai et al, (2011) J. Assoc. Res. Otolaryngol. 12: 455-469; and Shi et al., (2012) J. Neurosci. 32:9639-9648). Lgr5 is a known component of the Wnt/beta-catenin pathway, which has been shown to play major roles in differentiation, proliferation, and inducing stem cell characteristics (Barker et al. (2007) supra).

In the newborn cochlea, Lgr5+ cells showed the capacity to regenerate spontaneously after damage (Bramhall et al., (2014) Stem Cell Reports 2.311-322; and Cox et al., (2014) Development 141:816-829). But the spontaneous regeneration capacity was lost after the first postnatal week, and, indeed, no cell division or cell replacement occurs in the sensory epithelium of the adult mammal cochlea (Bramhall et al., (2014) supra; Cox et al., (2014) supra; Fujioka et al., (2015) Trends Neurosci. 38:139-141; Shi et al., (2012) supra).

Wnt proteins form a family of highly conserved secreted signaling molecules that regulate cell-to-cell interactions during embryogenesis. Wnt signaling becomes activated after damage and is known to function in the repair of tissue in several organ systems (i.e. Lim X, et al., 2013, Science 342:1226; Minear et al., (2010) Sci Transl Med. 2:29ra30). In the liver and pancreas, damage can elicit the emergence of Lgr5+ progenitor cells which are Wnt-responsive (i.e. Huch et al., (2013) Nature 494:247). In the inner ear, Lgr5+ WNT-responsive Supporting Cells (SCs) have been shown to be damage-recruited Hair Cell (HC) progenitors in neonatal mice (Wang et al., (2015) Nat Commun 6: 6613).

Recent research has revealed that a cocktail composed of growth factors, a WNT activator (e.g., GSK3), an HDAC inhibitor, and γ-secretase or Notch inhibitors (EGF, bFGF, IGF-1, CHIR, VPA, pVc, 616452, LY411575) can expand and differentiate Lgr5+ progenitor cells collected from adult mouse, monkey and human inner ear (McLean et al., (2017) Cell Reports 18:1917-1928). In this cocktail, the mechanism of WNT activation is indirect, via glycogen synthase kinase 3 (GSK3) inhibition.

Additionally, regulation of histone acetylation plays an important role in HC regeneration. In particular, histone acetyltransferases (HATs) enhance gene transcription while histone deacetylases (HDACs) function in the opposite direction, removing acetyl groups and repressing transcription. In ex vivo Corti explant models of ototoxicity, agonism of HATs and/or inhibition of HDACs (e.g., trichostatin-A, sodium butyrate) rescued HCs from ototoxic damage (see, e.g., Chen et al. (2009) J. Neurochem. 108:1226-1236. DNA methyltransferase (Dnmt) inhibitors (e.g., 5-azacytidine) have also demonstrated the ability to promote HC regeneration in models of ototoxicity (see, Deng, et al (2019) Scientific Reports 9:7997-7806).

The exploration of physiologic functions of WNT proteins in adult organisms has been hampered by functional redundancy and the necessity for conditional inactivation strategies. Dickkopf-1 (Dkk1) has been recently identified as the founding member of a family of secreted proteins that potently antagonize WNT signaling (see Glinka et al. (1998) Nature 391:357-62; Fedi et al. (1999) J Biol Chem 274:19465-72; and Bafico et al. (2001) Nat Cell Biol 3:683-6). Dkk1 associates with both the WNT co-receptors LRP5/6 and the transmembrane protein Kremen, with the resultant ternary complex engendering rapid LRP6 internalization and impairment of WNT signaling through the absence of functional Frizzled/LRP6 WNT receptor complexes (Mao et al. (2001) Nature 411:321-5; Semenov et al. (2001) Curr Biol 11:951-61; and Mao et al. (2002) Nature 417:664-7).

Exploration of WNT agonists has been hampered by the fact that they are not naturally soluble, diffusible molecules. The present invention provides methods to specifically modulate WNT signaling through particular FZD receptors with engineered soluble WNT agonists to achieve differential effect of epithelial regeneration. Clinical approaches combining WNT surrogates with other factors shows promise in treating hearing loss through HC regeneration via SC differentiation.

### I. Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, to tissues, to tissue organoids, and/or to organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and 30 additionally capable of being used in an animal to produce antibodies capable of binding to an epitope of that antigen. In certain embodiments, a binding agent (e.g., a WNT agonist molecule or binding region thereof) is said to specifically bind an antigen when it preferentially recognizes its target antigen in a complex mixture of proteins and/or macromolecules.

The term “antigen-binding fragment” as used herein refers to a polypeptide fragment that contains at least one CDR of an immunoglobulin heavy and/or light chain, or of a Nanobody® (Nab), that binds to the antigen of interest, in particular to one or more FZD receptors, or to LRP5 and/or LRP6. In this regard, an antigen-binding fragment of the herein described antibodies may comprise 1, 2, 3, 4, 5, or all 6 CDRs of a VH and VL from antibodies that bind one or more FZD receptors or LRP5 and/or LRP6.

As used herein, the terms “biological activity” and “biologically active” refer to the activity attributed to a particular biological element in a cell. For example, the “biological activity” of an WNT agonist or a tissue specific WNT signal enhancing molecule, or fragment or variant thereof refers to the ability to mimic or enhance WNT signals, respectively. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “epitope” is specific region on an antigen that an antibody recognizes and binds to, and is also referred to as the “antigenic determinant”. An epitope is usually 5-8 amino acids long on the surface of the protein. Proteins are three dimensionally folded structures, and an epitope may only be recognized in its form as it exists in solution, or its native form. When an epitope is made up of amino acids that are brought together by the three-dimensional structure, the epitope is conformational, or discontinuous. If the epitope exists on a single polypeptide chain, it is a continuous, or linear epitope. Depending on the epitope an antibody recognizes, it may bind only fragments or denatured segments of a protein, or it may also be able to bind the native protein.

The portion of an antibody or antibody fragment thereof that recognizes an epitope is referred to as the “epitope binding domain” or “antigen binding domain”. The epitope or antigen binding domain of an antibody or antibody fragment is in the Fab fragment and the effector functions in the Fc fragment. Six segments, known as complementarity determining regions (CDRs) within the variable regions (V_{H} and V_{L}) of the heavy and light chains loop out from the framework (FR regions) globular structure of the rest of the antibody and interact to form an exposed surface at one end of the molecule. This is the antigen binding domain. Generally, 4-6 of the CDRs will be directly involved in binding antigen, although fewer can provide the main binding motifs.

An “expression vector” is a vector, e.g. plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g. promoters, enhancers, UTRs, miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT agonist molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The term “native” or “wild-type” as used herein refers to a nucleotide sequence, e.g. gene, or gene product, e.g. RNA or protein, that is present in a wild-type cell, tissue, organ or organism. The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 50% or more, 60% or more, or 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the worldwide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

“RNA interference” as used herein refers to the use of agents that decrease the expression of a target gene by degradation of a target mRNA through endogenous gene silencing pathways (e.g., Dicer and RNA-induced silencing complex (RISC)). RNA interference may be accomplished using various agents, including shRNA and siRNA. “Short hair-pin RNA” or “shRNA” refers to a double stranded, artificial RNA molecule with a hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover. Small interfering RNA (siRNA) is a class of double-stranded RNA molecules, usually 20-25 base pairs in length, similar to miRNA, and operating within the RNA interference (RNAi) pathway. It interferes with the expression of specific genes with complementary nucleotide sequences by degrading mRNA after transcription, preventing translation. In certain embodiments, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)₂, Fv, single chain (scFv), Nanobodies® (Nabs), also known as VHH or single domain antibodies, DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., (1993) Proc. Natl. Acad. Sci. USA 90:6444-6448) are also a particular form of antibody contemplated herein. Minibodies comprising a scFv joined to a CH3 domain are also included herein (S. Hu et al., (1996) Cancer Res., 56:3055-3061; Ward, E. S. et al., (1989) Nature 341:544-546; Bird et al., (1988) Science, 242: 423-426, Huston et al., (1988) PNAS USA, 85: 5879-5883); PCT/US92/09965; WO94/13804; P. Holliger et al., (1993) Proc. Natl. Acad. Sci. USA 90 6444-6448; Y. Reiter et al., (1996) Nature Biotech, 14, 1239-1245; S. Hu et al., (1996) Cancer Res., 56, 3055-3061).

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule (Inbar et al. (1972) Proc. Nat. Acad. Sci. USA 69:2659-2662; Hochman et al. (1976) Biochem 15:2706-2710; and Ehrlich et al. (1980) Biochem 19:4091-4096).

In certain embodiments, single chain Fv or scFv antibodies are contemplated. For example, Kappa bodies (Ill et al., (1997) Prot. Eng. 10: 949-57); minibodies (Martin et al., (1994) EMBO J 13: 5305-9); diabodies (Holliger et al., (1993) PNAS 90: 6444-8); or Janusins (Traunecker et al., (1991) EMBO J 10: 3655-59 and Traunecker et al., (1992) Int. J. Cancer Suppl. 7: 51-52), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptors through one binding domain and to a second molecule through a second binding domain. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., (1989) Nature 341:544-546).

Where bispecific antibodies are to be used, these may be conventional bispecific antibodies, which can be manufactured in a variety of ways (Holliger, P. and Winter G., (1993) Current Opinion Biotechnol. 4:446-449), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., (1996) Protein Eng., 9: 616-621).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are

denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

Another antibody fragment contemplated is a dual-variable domain-immunoglobulin (DVD-Ig) is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al. (2007) Nature Biotechnology, 25:1290-1297).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al. (1988) PNAS 86:10029-10033; Riechmann et al. (1988) Nature 332:323-327). Illustrative methods for humanization of the anti-FZD or LRP antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

In certain embodiments, the antagonist or agonist binding agent binds with a dissociation constant (K_{D}) of about 1 μM or less, about 100 nM or less, about 40 nM or less, about 20 nM or less, or about 10 nM or less. For example, in certain embodiments, a FZD binding agent or antibody described herein that binds to more than one FZD, binds to those FZDs with a K_{D} of about 100 nM or less, about 20 nM or less, or about 10 nM or less. In certain embodiments, the binding agent binds to one or more its target antigen with an EC50 of about 1 μM or less, about 100 nM or less, about 40 nM or less, about 20 nM or less, about 10 nM or less, or about 1 nM 20 or less.

The antibodies or other agents of the present invention can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g. horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see e.g. Ausubel et al, eds, 1994, Current Protocols in Molecular Biology, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., FZD, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scatchard plot analysis. In some embodiments, BIAcore kinetic analysis is used to determine the binding on and off rates of antibodies or agents. BIAcore kinetic analysis comprises analyzing the binding and dissociation of antibodies from chips with immobilized antigens on their surface.

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

### II. General

The present invention provides methods of modulating WNT signals to ameliorate auditory disorders, including but not limited to, auditory or vestibular disorders arising from the destruction of cochlear hair cells, e.g., as a result of aging, injury from excessive noise, toxins, or a tumor. In particular embodiments, the present invention provides a WNT agonist to stimulate, increase, or enhance regeneration of cochlear hair cells.

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of WNT signaling pathways has potential for treatment of degenerative diseases and tissue injuries.

One of the challenges of developing a therapeutic WNT agonist for modulating WNT signaling, the existence of multiple WNT ligands and WNT receptors, Frizzled 1-10 (FZD1-10), with many tissues expressing multiple and overlapping FZDs. Canonical WNT signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to FZDs. In the inner ear, hair cell (HC) progenitors, also known as supporting cells (SC), express the leucine-rich repeat-containing receptor, Lgr5. The SCs act as undifferentiated precursor cells for HCs during hair cell regeneration (Shi, et al. (2012) J. Neurosci. 32:9639-9648). Recent studies have shown SCs expressing FZD9 could also differentiate into HCs as efficiently as Lgr5+ SCs (Zhang, et al. (2019) Front. Mol. Neurosci 12:184 (doi:10.3389/fnmo1.2019.00184)), implying that a FZD9 WNT agonist could be used to stimulate HC regeneration. The present invention provides WNT agonists specific for FZD9, as well as WNT agonists specific for FZDs1,2, and 7; FZDs5 and 8; FZD4; or FZD10.

R-spondins 1-4 are a family of ligands that amplify WNT signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson (2014) Matrix Biology 37: 157-161). R-spondins might also work through additional mechanisms of action. ZNRF3 and RNF43 are two membrane-bound E3 ligases specifically targeting WNT receptors (FZD1-10 and LRP5 or LRP6) for degradation. Binding of an R-spondin to ZNRF3/RNF43 and LGR4-6 causes clearance or sequestration of the ternary complex, which removes E3 ligases from WNT receptors and stabilizes WNT receptors, resulting in enhanced WNT signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying WNT signaling. While R-spondin effects depend on WNT signals, since both LGR4-6 and ZNRF3/RNF43 are widely expressed in various tissues, the effects of R-spondins are not tissue-specific.

### III. WNT Agonists

According to the present disclosure, in certain embodiments, a WNT agonist may be used for the treatment of auditory disorders. In particular, active WNT signaling can provide an Lgr5+SC proliferation signal and plays a key role in regulating regeneration of the cochlear cells in homeostasis and in injury. In particular embodiments, methods disclosed herein may be used to induce or increase: proliferation of supporting cells, such as, e.g., Lgr5-positive supporting cells; transdifferentiation of supporting cells into hair cells; expression of Atoh1, e.g., in supporting cells; the number of hair cells present in the inner ear of a subject.

In some embodiments, the WNT agonist can include binding agents or epitope binding domains that bind one or more FZD receptors and enhance WNT signaling. In certain embodiments, the agent or epitope binding domain (e.g., antibody) specifically binds to the cysteine-rich domain (CRD) within the human frizzled receptor(s) to which it binds. In some embodiments, the tissue targeted WNT signal enhancing molecule possesses binding agents or epitope binding domains that bind E3 ligases ZNRF3/RNF43. The E3 ligase agonist antibodies or fragments thereof can be single molecules or combined with other WNT antagonists, e.g., FZD receptor antagonists, LRP receptor antagonists, etc.

WNT agonists of the present invention are biologically active in binding to one or more FZD receptor and to one or more of LRP5 and LRP6, and in activation of WNT signaling, i.e., a WNT agonist. The term “WNT agonist activity” refers to the ability of an agonist to mimic the effect or activity of a WNT protein binding to a frizzled protein and/or LRP5 or LRP6. The ability of the WNT surrogate molecules and other WNT agonists disclosed herein to mimic the activity of WNT can be confirmed by a number of assays. WNT agonists typically initiate a reaction or activity that is similar to or the same as that initiated by the receptor's natural ligand. In particular, the WNT agonists disclosed herein activate, enhance or increase the canonical WNT/β-catenin signaling pathway. As used herein, the term “enhances” refers to a measurable increase in the level of WNT/β-catenin signaling compared with the level in the absence of a WNT agonist, e.g., a WNT surrogate molecule disclosed herein. In particular embodiments, the increase in the level of WNT/β-catenin signaling is at least 10%, at least 20%, at least 50%, at least two-fold, at least five-fold, at least 10-fold, at least 20-fold, at least 50-fold, or at least 100-fold as compared to the level of WNT/β-catenin signaling in the absence of the WNT agonist, e.g., in the same cell type. Methods of measuring WNT/β-catenin signaling are known in the art and include those described herein.

In particular embodiments, WNT agonists disclosed herein are bispecific, i.e., they specifically bind to two or more different epitopes, e.g., one or more FZD receptor, and LRP5 and/or LRP6. In certain embodiments the WNT surrogate molecules bind to FZDs1,2, and 7; FZDs5 and 8; FZD4; FZD9; or FZD10, and LRP5 and/or LRP6.

In particular embodiments, WNT agonists disclosed herein are multivalent, e.g., they comprise two or more regions that each specifically bind to the same epitope, e.g., two or more regions that bind to an epitope within one or more FZD receptor and/or two or more regions that bind to an epitope within LRP5 and/or LRP6. In particular embodiments, they comprise two or more regions that bind to an epitope within one or more FZD receptor and two or more regions that bind to an epitope within LRP5 and/or LRP6. In certain embodiments, WNT agonists comprise a ratio of the number of regions that bind one or more FZD receptor to the number of regions that bind LRP5 and/or LRP6 of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, WNT surrogate molecules are bispecific and multivalent.

In some embodiments the WNT agonist is an engineered recombinant polypeptide incorporating various epitope binding fragments that bind to various molecules in the WNT signaling pathway. For example, the FZD and LRP antibody fragments (e.g., Fab, scFv, VHH or sdAbs (a.k.a., Nanobodies®), etc) may be joined together directly or with various size linkers, on one molecule.

In certain embodiments the WNT agonist has a structure as described in WO2019126398 and WO2020010308. In one embodiment, the WNT agonist comprises two homodimeric chains, where each chain has at least one LRP 5 and/or 6 binding molecule which is a VHH or sdAb, attached by a linker to the N-terminus of a light chain of a Fab fragment that binds to at least one FZD receptor. The heavy chain of the Fab fragment is attached to an Ig domain thus making the entire construct resemble a full length antibody. In alternate embodiments the LRP 5/6 VHH can be attached to the N-terminus of the Fab heavy chain; the C-terminus of the Fab light chain; or the C-terminus of the Ig domain.

In one embodiment, the agonist molecule may also incorporate a tissue targeting moiety, e.g., an antibody or fragment thereof that recognizes a cochlear pillar cell- or a Deiter's cell-specific receptor or cell surface molecule.

### IV. Tissue-Specific WNT Signal Enhancing Molecules

According to the present disclosure, in certain embodiments, a tissue-specific WNT signal enhancing molecule may be used for the treatment of auditory disorders. The present disclosure provides novel tissue-specific WNT signal enhancing molecules capable of enhancing WNT activity in a tissue- or cell-specific manner. In certain embodiments, the tissue-specific WNT signal enhancing molecules are bi-functional molecules comprising a first domain that binds to one or more ZNRF3 and/or RNF43 ligases, and a second domain that binds to one or more targeted tissue or cell type in a tissue- or cell-specific manner. Each of the first domain and the second domain may be any moiety capable of binding to the ligase complex or targeted tissue or cell, respectively. For example, each of the first domain and the second domain may be, but are not limited to, a moiety selected from: a polypeptide (e.g., an antibody or antigen-binding fragment thereof or a peptide or polypeptide different from an antibody), a small molecule, and a natural ligand or a variant, fragment or derivative thereof. In certain embodiments, the natural ligand is a polypeptide, a small molecule, an ion, an amino acid, a lipid, or a sugar molecule. The first domain and the second domain may be the same type of moiety as each other, or they may be different types of moieties. In certain embodiments, the tissue-specific WNT signal enhancing molecules bind to a tissue- or cell-specific cell surface receptor. In particular embodiments, the tissue-specific WNT signal enhancing molecules increase or enhance WNT signaling by at least 50%, at least two-fold, at least three-fold, at least five-fold, at least ten-fold, at least twenty-fold, at least thirty-fold, at least forty-fold, or at least fifty-fold, e.g., as compared to a negative control.

Tissue-specific WNT signal enhancing molecules may have different formats. In particular embodiments, the tissue-specific WNT signal enhancing molecules are fusion proteins comprising a first polypeptide sequence that binds to ZNRF3/RNF43 and a second polypeptide sequence that binds to one or more targeted tissue or cell type in a tissue- or cell-specific manner. In certain embodiments, the two polypeptide sequences may be fused directly or via a linker. In certain embodiments, the tissue-specific WNT signal enhancing molecules comprise two or more polypeptides, such as dimers or multimers comprising two or more fusion proteins, each comprising the first domain and the second domain, wherein the two or more polypeptides are linked, e.g., through a linker moiety or via a bond between amino acid residues in each of the two or more polypeptides, e.g., an intermolecular disulfide bond between cysteine residues.

In particular embodiments, a tissue-specific WNT signal enhancing molecule is an antibody comprising antibody heavy and light chains (or antigen-binding fragments thereof) that constitute either the first domain or the second domain, wherein the other domain (i.e., the second domain or first domain) is linked to the antibody heavy chain or light chain, either as a fusion protein or via a linker moiety. In particular embodiments, the other domain is linked to the N-terminus of the heavy chain, the C-terminus of the heavy chain, the N-terminus of the light chain, or the C-terminus of the light chain. Such structures may be referred to herein as appended IgG scaffolds or formats. For example, a tissue-specific WNT signal enhancing molecule can be an antibody that binds ZNRF3/RNF43, wherein a binding domain that binds a tissue- or cell-specific receptor is fused or appended to either the heavy chain or light chain of the antibody that binds ZNRF3/RNF43. In another example, a tissue-specific WNT signal enhancing molecule can be an antibody that binds a tissue- or cell-specific receptor, wherein a binding domain that binds ZNRF3/RNF43 is fused or appended to either the heavy chain or light chain of the antibody that binds the tissue- or cell-specific receptor.

In particular embodiments, a tissue-specific WNT signal enhancing molecule specifically binds to a cochlear pillar cell or a Deiters' cell. In certain embodiments, the tissue-specific WNT signal enhancing molecule comprises an antibody or antigen-binding fragment thereof that binds p75NTR or FGFR3. In some embodiments, the tissue-specific WNT signal enhancing molecule comprises an antibody or antigen-binding fragment thereof that binds p75NTR or FGFR3, wherein a binding domain that binds ZNRF3/RNF43 is fused or appended to either the heavy chain or light chain of the antibody or antigen-binding fragment thereof. In particular embodiments, the binding domain that bind ZNRF3/RNF43 comprises Fu1 and Fu2 domains, wherein the Fu1 and Fu2 domains optionally comprise one or more amino acid modifications, including any of those disclosed herein, e.g., F105R and/or F109A.

In certain embodiments, the tissue-specific WNT signal enhancing molecules comprise a first domain (“action module”) that binds ZNRF3/RNF43 and a second domain (“targeting module”) that binds a tissue- or cell-specific receptor, e.g., with high affinity. In certain embodiments, the cell-specific receptor is p75NTR or FGFR3. In certain embodiments, each of these two domains has substantially reduced activity or is inactive in enhancing WNT signals by itself. However, when the tissue-specific WNT signal enhancing molecules engage with target tissues that express the tissue-specific receptor, E3 ligases ZNRF3/RNF43 are recruited to a ternary complex with the tissue-specific receptors, leading them to be sequestered, and/or cleared from the cell surface via receptor-mediated endocytosis. The net result is to enhance WNT signals in a tissue-specific manner.

In certain embodiments, the action module is a binder to ZNRF3/RNF43 E3 ligases, and it can be designed based on R-spondins, e.g., R-spondins-1-4, including but not limited to human R-spondins-1-4. In certain embodiments, the action module is an R-spondin, e.g., a wild-type R-spondin-1-4, optionally a human R-spondin-1-4, or a variant or fragment thereof. In particular embodiments, it is a variant of any of R-spondins-1-4 having at least 80%, at least 85%, at least 90%, at least 95%, at least 98%, or at least 99% sequence identity to the corresponding wild-type R-spondin-1-4 sequence. In certain embodiments, the action module comprises or consists of a Furin domain 1 of an R-spondin, e.g., any of R-spondins 1-4, which bind ZNRF3/RNF43. Extended versions of Furin domain 1 (including, but not limited to, those with a mutated Furin domain 2 that no longer binds to LGR4-6 or has reduced binding to LGR4-6) or engineered antibodies or any other derivatives or any engineered polypeptides different from antibodies that are able to bind specifically to ZNRF3/RNF43 can also be used. In certain embodiments, the action module comprises one or more Furin domain 1 of an R-spondin.

In certain embodiments, the action module does not comprise a Furin domain 2 of an R-spondin, or it comprises a modified or variant Furin domain 2 of an R-spondin, e.g., a Furin domain 2 with reduced activity as compared to the wild-type Furin domain 2. In certain embodiments, an action module comprises a Furin domain 1 but not a Furin domain 2 of R-spondin. In certain embodiments, an action module comprises two or more Furin domain 1 or multimers of a Furin domain 1. The action domain may comprise one or more wild-type Furin domain 1 of an R-spondin. In particular embodiments, the action module comprises a modified or variant Furin domain 1 of an R-spondin that has increased activity, e.g., binding to ZNRF3/RNF43, as compared to the wild-type Furin domain 1. Variants having increased binding to ZNRF3/RNF43 may be identified, e.g., by screening a phage or yeast display library comprising variants of an R-spondin Furin domain 1. Peptides or polypeptides unrelated to R-spondin Furin domain 1 but with increased binding to ZNRF3/RNF43 may also be identified through screening. Action modules may further comprise additional moieties or polypeptide sequences, e.g., additional amino acid residues to stabilize the structure of the action module or tissue-specific WNT signal enhancing molecule in which it is present.

In further embodiments, the action module comprises another inhibitory moiety, such as a nucleic acid molecule, which reduces or prevents ZNRF3/RNF43 activity or expression, such as, e.g., an anti-sense oligonucleotide; a small interfering RNA (siRNA); a short hairpin RNA (shRNA); a microRNA (miRNA); or a ribozyme. As used herein, “antisense” refers to a nucleic acid sequence, regardless of length, that is complementary to a nucleic acid sequence. In certain embodiments, antisense RNA refers to single-stranded RNA molecules that can be introduced to an individual cell, tissue, or subject and results in decreased expression of a target gene through mechanisms that do not necessarily rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA). In particular embodiments, the other inhibitor moiety inhibits an activity of one or both of ZNRF3/RNF43, or it inhibits the gene, mRNA or protein expression of one or both of ZNRF3/RNF43. In certain embodiments, the inhibitory moiety is a nucleic acid molecule that binds to a ZNRF3/RNF43 gene or mRNA, or a complement thereof.

In certain embodiments, the targeting module specifically binds to a cell-specific surface molecule, e.g., a cell-specific surface receptor, and can be, e.g., natural ligands, antibodies, or synthetic chemicals. In particular embodiments, the cell-specific surface molecule is preferentially expressed on a target organ, tissue or cell type, e.g., an organ, tissue or cell type in which it is desirous to enhance WNT signaling, e.g., to treat or prevent a disease or disorder. In particular embodiments, the cell-specific surface molecule has increased or enhanced expression on a target organ, tissue or cell type, e.g., an organ, tissue or cell type in which it is desirous to enhance WNT signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

Tissue-specific and cell-specific cell surface receptors are known in the art. Examples of tissue- and cell-specific surface receptors include but are not limited to p75NTR and FGFR3. In certain embodiments, the targeting module comprises an antibody or antigen-binding fragment thereof that specifically binds these intestine specific receptors.

In certain embodiments, a tissue-specific WNT signal enhancing molecule comprises may be one described, e.g., in WO2018140821 or WO2020014271.

In certain embodiments, components of the WNT surrogate and WNT signal enhancing molecules may be combined to confer more tissue specificity.

### V. WNT Agonist and Tissue Specific Signal Enhancing Combination Molecules

In certain embodiments it is advantageous to combine a WNT agonist described above with a tissue specific signal enhancing molecule. In one embodiment, the WNT agonist would comprise at least one LRP5/6 binding domain attached to at least one FZD binding domain. Also attached would be at least one binding domain that binds an E3 ubiquitin ligase (an antibody or fragment thereof that binds to ZNRF3 and or RNF43; an RSPO polypeptide; and at least one binding domain that binds a tissue specific receptor or cell surface molecule. Alternatively, the WNT agonist and tissue specific WNT signal enhancing molecule are separate molecules that could be administered in combination simultaneously or sequentially. In certain embodiments, a tissue-specific WNT signal enhancing combination molecule comprises any of the WNT agonists and/or any of the tissue-specific WNT signal enhancing molecules disclosed herein.

### VI. Pharmaceutical Compositions

The present disclosure also provides pharmaceutical compositions comprising a WNT agonist molecule, a tissue-specific WNT signal enhancing molecule, and/or a tissue-specific WNT signal enhancing combination molecule described herein and one or more pharmaceutically acceptable diluent, carrier, or excipient are also disclosed. In certain embodiments of methods disclosed herein, the WNT agonist molecule, tissue-specific WNT signal enhancing molecule, and/or tissue-specific WNT signal enhancing combination molecule are provided to the subject in one or more pharmaceutical composition.

In further embodiments, pharmaceutical compositions comprising a polynucleotide comprising a nucleic acid sequence encoding a WNT agonist molecule, a tissue-specific WNT signal enhancing molecule, or a tissue-specific WNT signal enhancing combination molecule described herein described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences.

In further embodiments, pharmaceutical compositions comprising an expression vector, e.g., a viral vector, comprising a polynucleotide comprising a nucleic acid sequence encoding a WNT agonist molecule, a tissue-specific WNT signal enhancing molecule, and/or a tissue-specific WNT signal enhancing combination molecule described herein and one or more pharmaceutically acceptable diluent, carrier, or excipient are also disclosed.

The present disclosure further contemplates a pharmaceutical composition comprising a cell comprising an expression vector comprising a polynucleotide comprising a promoter operatively linked to a nucleic acid encoding a WNT agonist molecule, tissue-specific WNT signal enhancing molecule, and/or tissue-specific WNT signal enhancing combination molecule, and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a WNT agonist. In particular embodiments, the cell is a heterologous cell or an autologous cell obtained from the subject to be treated.

The subject molecules, alone or in combination, can be combined with pharmaceutically acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the WNT agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a WNT agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al. (1977) “Pharmaceutical Salts,” J. Pharma Sci. 66:119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a WNT agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### VII. Methods of Use

The present disclosure also provides methods for using a WNT agonist molecule, a tissue-specific WNT signal enhancing molecule, and/or a tissue-specific WNT signal enhancing combination molecule, e.g., to modulate a WNT signaling pathway, e.g., to increase WNT signaling, and the administration of a WNT agonist molecule, a tissue-specific WNT signal enhancing molecule, and/or a tissue-specific WNT signal enhancing combination molecule in a variety of therapeutic settings. Provided herein are methods of treatment using a WNT agonist molecule, a tissue-specific WNT signal enhancing molecule, and/or a tissue-specific WNT signal enhancing combination molecule. In one embodiment, a WNT agonist molecule, a tissue-specific WNT signal enhancing molecule, and/or a tissue-specific WNT signal enhancing combination molecule is provided to a subject having a disease involving inappropriate or deregulated WNT signaling.

In certain embodiments, a WNT agonist molecule, a tissue-specific WNT signal enhancing molecule, and/or a tissue-specific WNT signal enhancing combination molecule may be used to enhance a WNT signaling pathway in a tissue or a cell. Agonizing the WNT signaling pathway may include, for example, increasing WNT signaling or enhancing WNT signaling in a tissue or cell. Thus, in some aspects, the present disclosure provides a method for agonizing a WNT signaling pathway in a cell, comprising contacting the tissue or cell with an effective amount of a WNT agonist molecule, a tissue-specific WNT signal enhancing molecule, and/or a tissue-specific WNT signal enhancing combination molecule, or a pharmaceutically acceptable salt thereof disclosed herein, wherein the WNT agonist molecule, tissue-specific WNT signal enhancing molecule, and/or tissue-specific WNT signal enhancing combination molecule is a WNT signaling pathway agonist. In some embodiments, contacting occurs in vitro, ex vivo, or in vivo. In particular embodiments, the cell is a cultured cell, and the contacting occurs in vitro.

The WNT agonist molecule, tissue-specific WNT signal enhancing molecule, and/or tissue-specific WNT signal enhancing combination molecule may be used for the treatment of auditory disorders, including but limited to, hearing loss caused by exposure to loud noise, aging, head trauma, virus infection, autoimmune inner ear disease, heredity, meniere's disease, otosclerosis, tumors, and vestibular disorders, including vestibular hypofunction.

In particular embodiments, the present invention provides a method for treating an auditory disorder in a subject in need thereof, comprising providing to the subject an effective amount of a WNT agonist molecule, tissue-specific WNT signal enhancing molecule, and/or tissue-specific WNT signal enhancing combination molecule. In particular embodiments, the WNT agonist molecule, tissue-specific WNT signal enhancing molecule, and/or tissue-specific WNT signal enhancing combination molecule is administered to the subject's ear, e.g., inner ear. In particular embodiments, the auditory disorder is hearing loss, e.g., hearing loss caused by exposure to loud noise, aging, ototoxicity, head trauma, virus infection, autoimmune inner ear disease, heredity, Meniere's disease, otosclerosis, tumors, or vestibular disorders, including vestibular hypofunction. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing or enhancing regeneration of damaged cochlear hair cells, including sensory hair cells, damaged as a result of injury from excessive noise, aging, or toxins, comprising providing to a subject in need thereof an effective amount of a WNT agonist molecule, tissue-specific WNT signal enhancing molecule, and/or tissue-specific WNT signal enhancing combination molecule. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing or enhancing regeneration of cochlear hair cells, e.g., sensory hair cells, in a subject's ear, comprising providing to a subject in need thereof an effective amount of a WNT agonist molecule, tissue-specific WNT signal enhancing molecule, and/or tissue-specific WNT signal enhancing combination molecule. In particular embodiments, the WNT agonist molecule, tissue-specific WNT signal enhancing molecule, and/or tissue-specific WNT signal enhancing combination molecule is administered to the subject's ear, e.g., inner ear. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing Atoh1 expression in supporting cells in a subject's ear, comprising providing to a subject in need thereof an effective amount of a WNT agonist molecule, tissue-specific WNT signal enhancing molecule, and/or tissue-specific WNT signal enhancing combination molecule. In particular embodiments, the WNT agonist molecule, tissue-specific WNT signal enhancing molecule, and/or tissue-specific WNT signal enhancing combination molecule is administered to the subject's ear, e.g., inner ear. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing proliferation of supporting cells in a subject's ear, comprising providing to a subject in need thereof an effective amount of a WNT agonist molecule, tissue-specific WNT signal enhancing molecule, and/or tissue-specific WNT signal enhancing combination molecule. In certain embodiments, the supporting cells comprise Lgr5+ supporting cells. In particular embodiments, the WNT agonist molecule, tissue-specific WNT signal enhancing molecule, and/or tissue-specific WNT signal enhancing combination molecule is administered to the subject's ear, e.g., inner ear. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing transdifferentiation of supporting cells into hair cells in a subject's ear, comprising providing to a subject in need thereof an effective amount of a WNT agonist molecule, tissue-specific WNT signal enhancing molecule, and/or tissue-specific WNT signal enhancing combination molecule. In particular embodiments, the WNT agonist molecule, tissue-specific WNT signal enhancing molecule, and/or tissue-specific WNT signal enhancing combination molecule is administered to the subject's ear, e.g., inner ear. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In particular embodiments, the present invention provides a method for increasing the number of hair cells, e.g., sensory hair cells, in a subject's ear, comprising providing to a subject in need thereof an effective amount of a WNT agonist molecule, tissue-specific WNT signal enhancing molecule, and/or tissue-specific WNT signal enhancing combination molecule. In particular embodiments, the WNT agonist molecule, tissue-specific WNT signal enhancing molecule, and/or tissue-specific WNT signal enhancing combination molecule is administered to the subject's ear, e.g., inner ear. In certain embodiments, the subject is administered a WNT agonist molecule. In certain embodiments, the subject is administered a tissue-specific WNT signal enhancing molecule. In certain embodiments, the subject is administered a WNT agonist molecule and a tissue-specific WNT signal enhancing molecule. In certain embodiments, the subject is administered a tissue-specific WNT signal enhancing combination molecule.

In one embodiment, the WNT agonist molecule may also incorporate a tissue targeting moiety, e.g., an antibody or fragment thereof that recognizes a cochlear pillar cell- or a Deiter's cell-specific receptor or cell surface molecule.

In certain embodiments, the WNT agonist and tissue specific WNT signal enhancing molecule can be combined to enhance the regeneration of the cochlear hair cells. The WNT agonist and WNT signal enhancing molecule can be on multiple molecules or on one molecule (e.g., a tissue-specific WNT signal enhancing combination molecule).

The present invention also provides for combination treatment with another compound for hair cell regeneration in vitro. For example, the WNT agonist and/or tissue-specific WNT signal enhancing molecule, or tissue-specific WNT signal enhancing combination molecule can be used in combination with another molecule to promote hair cell regeneration in vitro. Such other molecules may include one or more of, but are not limited to: growth factors, such as epidermal growth factor (EGF), basic fibroblast growth factor (bFGF), and insulin like growth factor 1 (IGF-1); histone acetylase agonists; histone acetyl transferase (HAT) agonists, DNA methyl transferase (DNMT) inhibitors, histone deacetylase (HDAC) inhibitors, such as valproic acid; 2-phosph-L-ascorbic acid (pVc); Notch signal modulators, such as γ-secretase inhibitors (see, e.g., Golde, et al. (2013) Biochim Biophys. Acta 1828:1-27); and TGF-β inhibitors, such as an Alk5 inhibitor (616452). The WNT agonist, tissue specific WNT signal enhancer, or a combination of both may be administered with a cocktail of EGF, bFGF, IGF-1, valproic acid, pVc and 616452, collectively known as EFIVP6 cocktail (see, e.g., McLean et al. (2017) Cell Reports 18:1917-1929).

The WNT agonist and/or tissue specific WNT signal enhancer, or tissue-specific WNT signal enhancing combination molecule, can also be combined with a viral vector delivered Atoh1 (Atonal transcription factor 1) gene therapy. For example, Ad-Atoh1, also known as CGF166, can be locally delivered to the cochlea with the WNT agonists and/or tissue specific WNT signal enhancers of the current invention. The above compounds can be administered sequentially or concurrently with the molecules of the present invention.

The therapeutic agent (e.g., a WNT agonist) may be administered before, during or after the onset of disease or injury. The treatment of ongoing disease, where the treatment stabilizes or reduces the undesirable clinical symptoms of the patient, is of particular interest. Such treatment is desirably performed prior to or after a complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. In the present invention, the therapeutic benefit may be measured by an increase in expression of HC markers such as Myosin VIIa and/or F-actin (see, e.g., Deng et al (2019) supra). Another measure of therapeutic benefit can be a standard hearing test. The ordinarily skilled artisan will appreciate that such measures of therapeutic efficacy will be applicable to the particular disease being modified, and will recognize the appropriate detection methods to use to measure therapeutic efficacy.

Administration of the WNT agonist and/or WNT signal enhancer, or tissue-specific WNT signal enhancing combination molecule, may be accomplished systemically by intravenous or subcutaneous injection. Alternatively, the WNT agonist and/or WNT signal enhancer, or tissue-specific WNT signal enhancing combination molecule, may be delivered locally to the inner ear by a variety of methods including injection (see, e.g., Kechai, et al. (2015) Int. J. Pharmaceutics 494:83-101). Large proteins, like monoclonal antibodies similar in size and biochemical composition, were previously shown to diffuse through the round window making this approach feasible for engineered WNT agonists and tissue specific WNT signal enhancers (Ghossaini et al. (2013) Laryngoscope. 123, 2840-2844). In addition, WNT agonists and/or WNT signal enhancers, or tissue-specific WNT signal enhancing combination molecules, may be administered by other local delivery approaches including, but not limited to, direct delivery into inner ear.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this invention is best understood with reference to the following examples, which are not intended to limit the inventions to the specific embodiments.

### Examples

### I. General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Expression of Frizzled Receptors in Adult Cochlea

The expression pattern of each of the Frizzled receptors in the mouse cochlea, was determined by in situ hybridization.

mRNA for individual FZD receptors, LRP5 and LRP6 co-receptors, and Axin2 were detected by in situ hybridization. RNAscope® probes (ACD) used are listed in Table 1. Standard RNAscope® 2.5 HD Assay-Red protocol was followed.

TABLE 1

| ACD | |
|---|---|
| catalogue # | Probes |
| 404871 | RNAscope ® Probe-Mm-FZD1 |
| 404881 | RNAscope ® Probe-Mm-FZD2 |
| 404891 | RNAscope ® Probe-Mm-FZD3 |
| 404901 | RNAscope ® Probe-Mm-FZD4 |
| 404911 | RNAscope ® Probe-Mm-FZD5 |
| 404921 | RNAscope ® Probe-Mm-FZD6 |
| 404931 | RNAscope ® Probe-Mm-FZD7 |
| 404941 | RNAscope ® Probe-Mm-FZD8 |
| 404951 | RNAscope ® Probe-Mm-FZD9 |
| 315781 | RNAscope ® Probe-Mm-FZD10 |
| 315791 | RNAscope ® Probe-Mm-Lrp5 |
| 315801 | RNAscope ® Probe-Mm-Lrp6 |
| 443610 | RNAscope ® Probe-Mm-Axin2 |

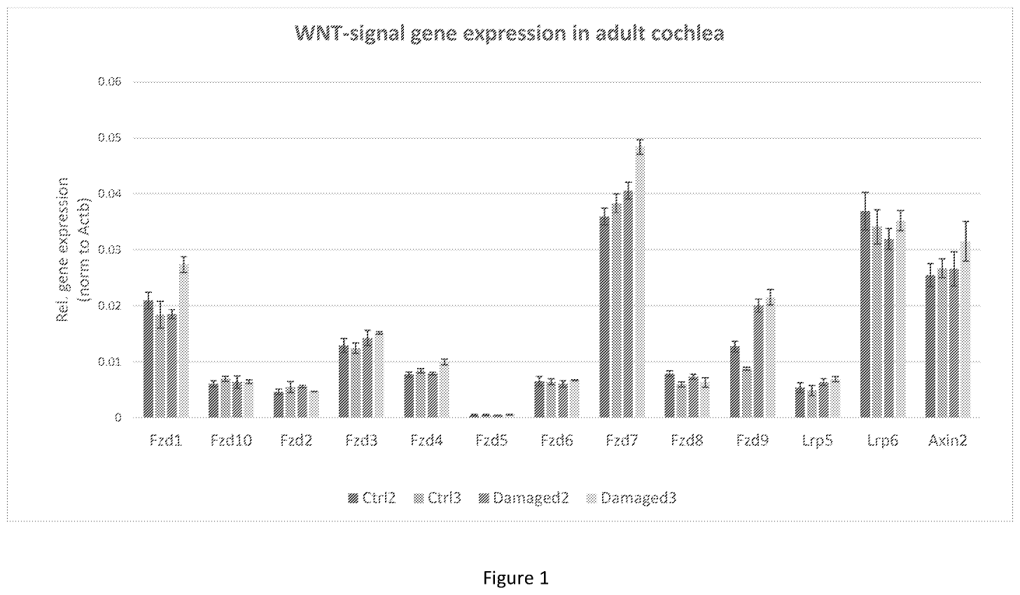

FZD1, FZD2, FZD3, FZD4, FZD5, FZD6, FZD7, FZD8, FZD9, and FZD10 mRNA are expressed at different levels in the mouse cochlea (FIG. 1). FZD1, 3, 7, and 9 were expressed at the highest level. Further, FZD9 expression was significantly induced by tissue damage (FIG. 1). LRP5 and LRP6 mRNA were both expressed, with Lrp6 expressed at the highest level. Expression of Axin2 mRNA demonstrated active WNT signaling in this tissue.

### III. Activities of Engineered Soluble WNT Agonists and WNT Signal Enhancer

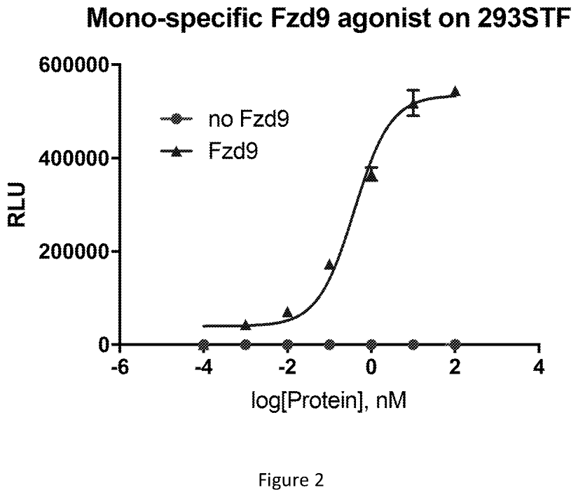

Human Embryonic Kidney Cells 293 (HEK293) STF reporter cells (see, e.g., Fuerer and Nusse (2010) PLoS One 5:39370) were used to measure the response to engineered WNT agonist stimulation (see, e.g., FIG. 2). Reporter gene expression was stimulated by the WNT agonist clone, 14SB6-26, a mono-specific FZD9 agonist in cells ectopically expressing the FZD9 receptor. Other WNT agonists, including those disclosed in Table 2, are also tested in STF reporter cells expressing relevant FZD receptors. Each of the WNT agonists comprises a heavy chain (HC) and a light chain (LC) as disclosed in Table 2.

TABLE 2
Sequences of WNT Agonist Clones

| WNT | SEQ ID | |
|---|---|---|
| AGONIST | NO: | SEQUENCE |
| R2M3-26 HC | 1 | 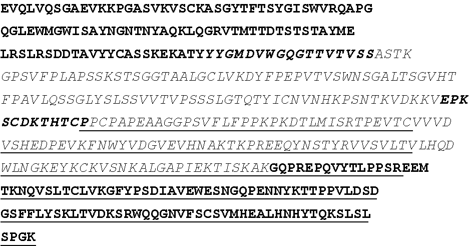 |
| R2M3-26 LC | 2 | 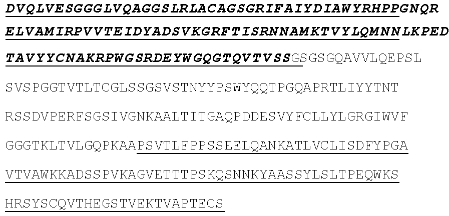 |
| 1RC07-26 HC | 3 | QVQLQQWGAGLLKPSETLSLTCAVSGASFSGHYWTWIRQPP |
| | | GKGLEWIGEIDHTGSTNYEPSLRSRVTISVDTSKNQFSLNLKSV |
| | | TAADTAVYYCARGGQGGYDWGHYHGLDVWGQGTTVTVSSA |
| | | STKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCVVV |
| | | DVSHEDPEVKFNWYVDGVEVHNAKTKPREEQYNSTYRVVSVLTVLHQ |
| | | DWLNGKEYKCKVSNKALGAPIEKTISKAKGQPREPQVYTLPPSREE |
| | | MTKNQVSLTCLVKGFYPSDIAVEWESNGQPENNYKTTPPVLD |
| | | SDGSFFLYSKLTVDKSRWQQGNVFSCSVMHEALHNHYTQKSL |
| | | SLSPGK |
| 1RC07-26 LC | 4 | 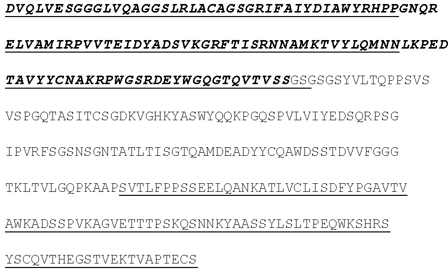 |
| R2M13-26 HC | 5 | EVQLLQSGAEVKKPGSSVKVSCKASGGTFTYRYLHWVRQAPG |
| | | QGLEWMGGIIPIFGTGNYAQKFQGRVTITADESTSTAYMELSS |
| | | LRSEDTAVYYCASSMVRVPYYYGMDVWGQGTLVTVSSASTKG |
| | | PSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSC |
| | | DKTHTCPPCPAPEAAGGPSVFLFPPKPKDTLIVIISRTPEVTCVVVDVSH |
| | | EDPEVKFNWYVDGVEVHNAKTKPREEQYNSTYRVVSVLTVLHQDWLN |
| | | GKEYKCKVSNKALGAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFSC |
| | | SVMHEALHNHYTQKSLSLSPGK |
| R2M13-26 LC | 6 | 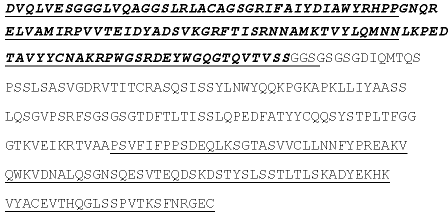 |
| 4SD1-3 HC | 7 | 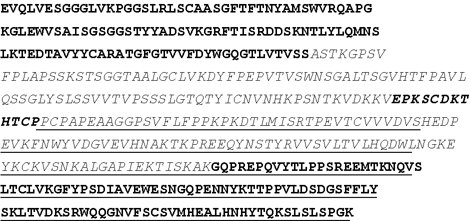 |
| 4SD1-3 LC | 8 | 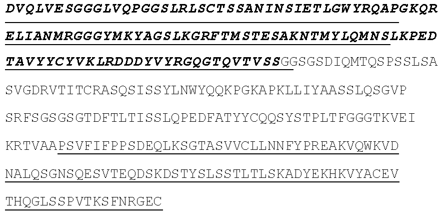 |
| 14SB6-26 HC | 9 | 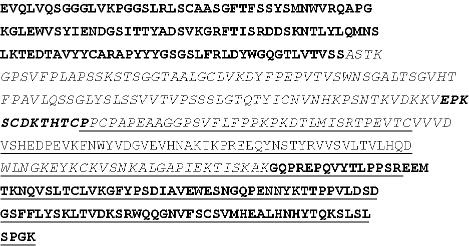 |
| 14SB6-26 LC | 10 | 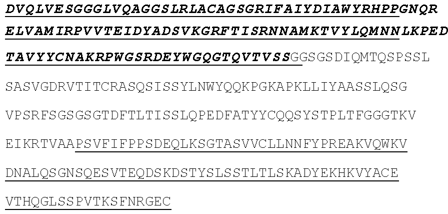 |
| 5SE12-26 HC | 11 | 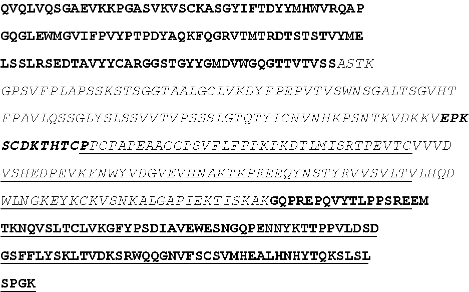 |
| 5SE12-26 LC | 12 | 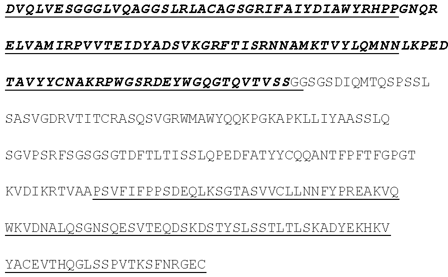 |

The FZD-VH sequences are indicated in bold; the FZD-CH1 sequences are indicated in italics; the hinge sequences are indicated in bold italics; the CH2 sequences are indicated in underlined italics; the CH3 sequences are indicated in bold underline; the LRP VHH sequence, which is attached to the N-terminus of the VL via a linker, is indicated in bold italic underline; the linker sequence is underline only; the FZD-VL is shaded gray; and the FZD-CL is shaded gray and underlined.

The activity of the Frizzled biased WNT agonists, for example, R2M3-26 and R2M3-3 (FZD1,2,5,7,8), 1RC07-26 and 1RC07-3 (FZD1,2,7) and R2M13-26 (FZD5,8), mono Frizzled-specific WNT agonists, 4SD1-3 (FZD4), 14SB6-26 and 14SB6-3 (FZD9), 5SE12-26 (FZD10), as well as tissue-specific WNT signal enhancers, (i.e. an antibody that binds p75NTR or FGFR3 fused with a binding domain for ZNRF3/RNF43) are examined using this ex-vivo primary culture assays of mouse cochlear and vestibular tissues (neonates and adults) (+/−prior neomycin damage). anti-eGFP control protein may be used in parallel as a control treatment. Additional WNT agonists may be used and are described, e.g., in WO2019126398 and WO2020010308. Tissue targeted WNT signal enhancers may also be used and are described, e.g., in WO2018140821 and WO2020014271.

Titrations of engineered WNT agonists are performed for optimization of dose response and assessment of inner ear regeneration (proliferation and new hair cell formation). This ex-vivo assay is used to determine the proper dose to stimulate HC regeneration with and without addition of EFIVP6 cocktail (EGF, bFGF, IGF-1 (EFI); EFI and VPA (HDAC inhibitor), pVc (2-phospho-L-ascobic acid), 616452 (TGF-β receptor (Alk5) inhibitor; McLean et al. (2017) Cell Reports 18:1917-1928). Increases in SCs and HC are assessed histologically.

### IV. Activity of Engineered WNT Agonists on HC Regeneration In Vivo

Wnt signaling has been shown to be an important component for hair cell regeneration in mouse models. An ex-vivo model with cells derived from both neonatal and adult mouse cochlea in a Matrigel-based 3D culture system is used to test for activity of these soluble WNT agonists. Previous studies have used an inhibitor of the WNT negative regulator, glycogen-synthase kinase 3 (CHIR99021; Mclean et al. (2017) Cell Reports 18:1917-1928), together with other factors to induce growth of support cells (SCs), and to induce their trans-differentiation into new hair cells (HCs). Soluble, engineered WNT agonists are used in place of CHIR99021.

Engineered WNT agonists, tissue specific WNT signal enhancers, and control proteins (e.g., GFP) are locally applied using the round window application method (Kechai et al., (2015) Int. J. Pharm. 494:83-101) to the inner ear in rodent models (e.g., mouse, rat, or guinea pig). Both histological and physiological responses of the rodent utricle and/or cochlea are assessed. Treatment is done with WNT agonists and/or tissue targeted WNT enhancers in EFIVP6 cocktail (WNT agonists and/or tissue targeted WNT enhancers added instead of CHIR99021); EFICVP6 cocktail from McLean et al. (2017) Cell Reports 18:1917-1928.

After damage (via, e.g., neomycin, noise, or other auditory insults) induction in rodents, local injection of WNT agonist molecules, tissue targeted WNT enhancer molecules, WNT agonist-EFIVP6 cocktail, WNT agonist plus tissue targeted WNT enhancer combination, tissue targeted WNT enhancer-EFIVP6 cocktail, or WNT agonist-EFIVP6 cocktail-tissue targeted WNT enhancer combination are used to test the efficacy of each combination in rodent models.

Activation of WNT signaling specifically through FZD stimulated HC regeneration in vivo can be measured by both histology to assess HC regeneration and by a traditional hearing testing to measure the physiological endpoint of hearing conduction.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. All references cited herein are incorporated by reference to the same extent as if each individual publication, patent application, or patent, was specifically and individually indicated to be incorporated by reference. All publications mentioned herein are incorporated herein by reference to disclose and describe the methods and/or materials in connection with which the publications are cited. It is understood that the present disclosure supersedes any disclosure of an incorporated publication to the extent there is a contradiction.

Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of treating a subject suffering from an auditory disorder comprising administering the subject, an engineered WNT signaling modulator.

2. The method of claim 1 wherein the WNT signaling modulator is an engineered WNT agonist.

3. The method of claim 1, wherein the engineered WNT agonist is selected from the group consisting of an engineered polypeptide, an engineered antibody containing at least one epitope binding domain, a small molecule, an siRNA, and an antisense nucleic acid molecule.

4. The method of claim 2, wherein the engineered WNT agonist comprises binding compositions that bind to one or more FZD receptors and binding compositions that bind to one or more LRP receptors.

5. The method of any of claims 1-4, wherein the engineered WNT agonist comprises a tissue targeting molecule.

6. The method of claim 5, wherein the tissue targeting molecule is an antibody or fragment thereof that binds to a tissue specific cell surface antigen.

7. The method of any of claims 1-6 wherein the WNT agonist is administered with another molecule selected from the group consisting of growth factors, HDAC inhibitors, and gamma-secretase inhibitors.

8. The method of claim 1, wherein the auditory disease is hearing loss due to cochlear tissue damage.

9. The method of claim 8, wherein the cochlear tissue damage is sensory hair cell loss.

10. A method of treating a subject suffering from an auditory disorder comprising administering the subject, a tissue-specific WNT signal enhancing molecule.

11. The method of claim 10, wherein the WNT signal enhancing molecule is an engineered molecule comprising:
a. a first domain that binds to one or more E3 ubiquitin ligases; and
b. a second domain that binds to a tissue specific receptor.

12. The method of claim 11, wherein the E3 ubiquitin ligases are selected from the group consisting of Zinc and Ring Finger Protein 3 (ZNRF3) and Ring Finger Protein 43 (RNF43).

13. The method of claim 11, wherein the first domain comprises an R-spondin (RSPO) polypeptide.

14. The method of claim 13, wherein the RSPO polypeptide is selected from the group consisting of RSPO-1, RSPO-2, RSPO-3, and RSPO-4.

15. The method of claim 13, wherein the RSPO polypeptide comprises a first furin domain and a second furin domain.

16. The method of claim 15, wherein the second furin domain is wild-type or is mutated to have lower binding to Leucine-rich repeat-containing G protein coupled receptors 4-6 (LGR4-6).

17. The method of claim 10, wherein the WNT signal enhancing molecule incorporates a tissue targeting molecule.

18. The method of claim 17, wherein the tissue targeting molecule is an antibody or fragment thereof that binds to a tissue specific cell surface antigen.

19. The method of any of claims 10-18, wherein the WNT agonist is administered locally by injection into an inner ear.

20. The method of any of claims 10-19 wherein the WNT agonist is administered with another molecule selected from the group consisting of growth factors, HDAC inhibitors, gamma-secretase inhibitors, and Notch signal modulators.

21. The method of claim 10, wherein the auditory disorder is selected from the group consisting of: for the treatment of auditory disorders, including but limited to, hearing loss caused by exposure to loud noise, aging, ototoxicity, head trauma, virus infection, autoimmune inner ear disease, heredity, Meniere's disease, otosclerosis, tumors, and vestibular disorders, including vestibular hypofunction.

22. The method of any of claim 21, wherein the auditory disorder is hearing loss due to cochlear tissue damage.

23. The method of claim 22, wherein cochlear tissue damage is loss of sensory hair cells.

24. A method of treating a subject suffering from an auditory disorder comprising administering the subject, an engineered WNT agonist and an engineered tissue specific WNT signal enhancing combination molecule.

25. The method of claim 24, wherein the combination molecule comprises:
a. the engineered WNT agonist having at least one binding domain that binds to at least one FZD receptor and at least one binding domain that binds to at least one LRP receptor; and
b. the engineered WNT signal enhancing molecule comprising a first domain that binds to one or more E3 ubiquitin ligases; and a second domain that binds to a tissue specific receptor.

26. The method of claim 25, wherein the E3 ubiquitin ligases are selected from the group consisting of Zinc and Ring Finger Protein 3 (ZNRF3) and Ring Finger Protein 43 (RNF43).

27. The method of claim 26, wherein the first domain comprises an R-spondin (RSPO) polypeptide.

28. The method of claim 27, wherein the RSPO polypeptide is selected from the group consisting of RSPO-1, RSPO-2, RSPO-3, and RSPO-4.

29. The method of claim 27, wherein the RSPO polypeptide comprises a first furin domain and a second furin domain.

30. The method of claim 29, wherein the second furin domain is wild-type or is mutated to have lower binding to Leucine-rich repeat-containing G protein coupled receptors 4-6 (LGR4-6).

31. The method of claim 24, wherein the combination molecule incorporates a tissue targeting molecule.

32. The method of claim 31, wherein the tissue targeting molecule is an antibody or fragment thereof that binds to a tissue specific cell surface antigen.

33. The method of any of claims 24-32, wherein the combination molecule is administered locally by injection into an inner ear.

34. The method of any of claims 24-32 wherein the WNT agonist is administered with another molecule selected from the group consisting of growth factors, HDAC inhibitors, and gamma-secretase inhibitors.

35. The method of claim 24, wherein the auditory disorder is selected from the group consisting of: for the treatment of auditory disorders, including but limited to, hearing loss caused by exposure to loud noise, aging, ototoxicity, head trauma, virus infection, autoimmune inner ear disease, heredity, Meniere's disease, otosclerosis, tumors, and vestibular disorders, including vestibular hypofunction.

36. The method of claim 24, wherein the auditory disorder is hearing loss due to cochlear tissue damage.

37. The method of claim 36, wherein the cochlear tissue damage is loss of sensory hair cells.


SEQUENCE LISTING (12 sequences)

SEQ ID NO: 1 - protein, 452 aa, Artificial Sequence
  Made in Lab - Engineered WNT agonist domain
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 340 aa, Artificial Sequence
  Made in Lab - Engineered WNT agonist domain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 454 aa, Artificial Sequence
  Made in Lab - Engineered WNT agonist domain
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 337 aa, Artificial Sequence
  Made in Lab - Engineered WNT agonist domain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 452 aa, Artificial Sequence
  Made in Lab - Engineered WNT agonist domain
EVQLLQSGAEVKKPGSSVKVSCKASGGTFTYRYLHWVRQAPGQGLEWMGGIIPIFGTGNYAQKFQGRVTITADESTSTAYMELSSLRSEDTAVYYCASSMVRVPYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 341 aa, Artificial Sequence
  Made in Lab - Engineered WNT agonist domain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGSGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, Artificial Sequence
  Made in Lab - Engineered WNT agonist domain
EVQLVESGGGLVKPGGSLRLSCAASGFTFTNYAMSWVRQAPGKGLEWVSAISGSGGSTYYADSVKGRFTISRDDSKNTLYLQMNSLKTEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 336 aa, Artificial Sequence
  Made in Lab - Engineered WNT agonist domain
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 454 aa, Artificial Sequence
  Made in Lab - Engineered WNT agonist domain
EVQLVQSGGGLVKPGGSLRLSCAASGFTFSSYSMNWVRQAPGKGLEWVSYIENDGSITTYADSVKGRFTISRDDSKNTLYLQMNSLKTEDTAVYYCARAPYYYGSGSLFRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 338 aa, Artificial Sequence
  Made in Lab - Engineered WNT agonist domain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGSGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Artificial Sequence
  Made in Lab - Engineered WNT agonist domain
QVQLVQSGAEVKKPGASVKVSCKASGYIFTDYYMHWVRQAPGQGLEWMGVIFPVYPTPDYAQKFQGRVTMTRDTSTSTVYMELSSLRSEDTAVYYCARGGSTGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 338 aa, Artificial Sequence
  Made in Lab - Engineered WNT agonist domain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGSGSDIQMTQSPSSLSASVGDRVTITCRASQSVGRWMAWYQQKPGKAPKLLIYAASSLQSGVPSRFSGSGSGTDFTLTISSLQPEDFATYYCQQANTFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC